(19) United States
(12) Patent Application Publication
(10) Pub. No.: US 2022/0392633 A1
(43) Pub. Date: Dec. 8, 2022

(54) PERSONALIZED DATA GRAPHS INCLUDING USER DOMAIN CONCEPTS

(71) Applicant: Apple Inc., Cupertino, CA (US)

(72) Inventors: Lucas B. Spicer, San Jose, CA (US); Zhe Li, Santa Monica, CA (US); Joefrey S. Kibuule, San Francisco, CA (US); Eric J. Mertz, Santa Clara, CA (US); Jamal E. Kharrat, San Jose, CA (US); David T. Wilson, Cupertino, CA (US)

(73) Assignee: Apple Inc., Cupertino, CA (US)

(21) Appl. No.: 17/831,730

(22) Filed: Jun. 3, 2022

Related U.S. Application Data
(60) Provisional application No. 63/197,476, filed on Jun. 6, 2021.

(51) Int. Cl. G16H 40/67; G16H 10/60

(52) U.S. Cl. CPC G16H 40/67; G16H 10/60

(57) ABSTRACT

A user domain concept (UDC) node of a personalized data graph may be generated by a user device. The UDC node may include customized information that represents some user-customized portion of health data, e.g., data obtained from an electronic health record (EHR) system. The UDC node may be generated based on a customization request, e.g., a request from a user to customize the portion of the health data and/or other aspects. The UDC node may store relationship information between nodes and be used to improve the functioning of a user interface that presents the health data. This may be achieved, at least in part, by providing user customization of the user interface (e.g., favoriting records, adding records to lists, giving records nicknames).

### CROSS-REFERENCES TO RELATED APPLICATIONS

This application claims priority under 35 U.S.C. § 119(e) to U.S. Provisional Application No. 63/197,476, filed on Jun. 6, 2021 entitled, “PERSONALIZED DATA GRAPHS INCLUDING USER DOMAIN CONCEPTS,” the contents of which are herein incorporated by reference.

### BACKGROUND

Advancements have been made in recent years that allow users to download health data from remote electronic health record (EHR) systems to their mobile user devices. For example, a user may use a smartphone to connect to an endpoint of the EHR system and download a copy of their clinical health record. The smartphone may include an application configured to process and organize health data present in the clinical health record.

### BRIEF SUMMARY

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a computer-implemented method. The computer-implemented method includes receiving, by a user device, health data from an electronic health record (EHR) system. The computer-implemented method also includes receiving a customization request relating to customizing a portion of the health data. The computer-implemented method also includes generating a user domain concept (UDC) node of a personalized data graph based at least in part on the customization request, the UDC node including customized information that represents the customized portion of the health data. The computer-implemented method also includes updating, based at least in part on generating the UDC node, the UDC node to include relationship information that represents a relationship between the UDC node and the portion of the health data. The computer-implemented method also includes populating, at the user device, a user interface that includes a representation of the UDC node. Other embodiments of this aspect include corresponding computer systems, apparatus, and computer programs recorded on one or more computer storage devices, each configured to perform the actions of the methods.

One general aspect includes a computer-implemented method. The computer-implemented method includes receiving, by a user device, health data from an electronic health record (EHR) system. The computer-implemented method also includes generating a concept node of a personalized data graph by at least determining a mapping between a medical concept identified from the health data and a reference node of a reference ontology. The computer-implemented method also includes receiving a customization request relating to customizing an aspect of the concept node. The computer-implemented method also includes generating a user domain concept (UDC) node of the personalized data graph based at least in part on the customization request, the UDC node including customized information that represents the customized aspect of the concept node. The computer-implemented method also includes updating, based at least in part on generating the UDC node, the concept node to include relationship information that represents a relationship between the concept node and the UDC node. The computer-implemented method also includes populating, at the user device, a user interface that includes a representation based at least in part on the UDC node. Other embodiments of this aspect include corresponding computer systems, apparatus, and computer programs recorded on one or more computer storage devices, each configured to perform the actions of the methods.

### BRIEF DESCRIPTION OF THE DRAWINGS

FIG. 1 illustrates a block diagram and a flowchart showing an example process for generating user domain concept nodes of personalized data graphs, according to at least one example.

FIG. 2 illustrates a block diagram showing an example architecture or system for enabling techniques relating to generating user domain concept nodes of personalized data graphs, according to at least one example.

FIG. 3 illustrates a diagram showing example user configurations for storing health data on a user device relating to user domain concept nodes of personalized data graphs, according to at least one example.

FIG. 4 illustrates a block diagram showing an example data model for generating user domain concept nodes of personalized data graphs, according to at least one example.

FIG. 5 illustrates a flow chart showing an example process for generating user domain concept nodes of personalized data graphs, according to at least one example.

FIG. 6 illustrates a flow chart showing an example process for generating user domain concept nodes of personalized data graphs, according to at least one example.

FIG. 7 illustrates an example architecture or environment configured to implement techniques described herein, according to at least one example.

### DETAILED DESCRIPTION

In the following description, various examples will be described. For purposes of explanation, specific configurations and details are set forth in order to provide a thorough understanding of the examples. However, it will also be apparent to one skilled in the art that the examples may be practiced without the specific details. Furthermore, well-known features may be omitted or simplified in order not to obscure the example being described.

Examples of the present disclosure are directed to, among other things, methods, systems, devices, and computer-readable media that generate and securely store user domain concepts (UDC) relating to health data. The UDCs may be stored as a node in a node-based relational storage structure. Thus, in some examples, a UDC may be referred to as a UDC node. Generally, a UDC may be a user-configurable abstraction around a health concept (e.g., a health concept identified from the health data) or an abstraction around such health concepts. For example, a smartphone application may enable a user to customize certain aspects of their health data that have been downloaded on to the user's smartphone, and these customizations may be saved using UDCs.

The use of UDCs provides for a structured manner to store and retain customizable mutable user data that can be separately associated with both reference concepts of a reference ontology (so they can be updated by content experts) and medical records (which are generally immutable except by the healthcare institutions). Thus, the UDCs provide a mechanism for bridging the gap between the immutable medical record data and the reference ontology that may be periodically updated as more content becomes available and relevant. This mechanism provides for an improved user experience as it relates to customizing, understanding, and efficiently viewing their health data on their user device. This improved experience may be realized at least in part because fewer selections, click-throughs, etc. may be required to access certain health data. For example, a “pinned” or “favorited” medication (as enabled by the UDCs) may be available to a user on a home screen (or landing page) of the user interface of an application, rather than the user having to search at multiple different possible locations within the application.

The use of UDCs may enable improved persistence of user customizations even when the health data upon which the customizations are built and/or reference ontologies upon which the customizations are based are changed over time. The UDCs may also be persisted across multiple devices that are associated with the same user profile of the user (e.g., the user has logged into a smartphone, a watch, and a tablet using the same user profile) in a manner currently unavailable. A UDC may include some amount of instance user configuration state (e.g., favorited, nickname, present in a given list, etc.), can be connected to underlying health data (e.g., health data stored on the user device), can be connected to other UDCs, can be connected to an underlying reference ontology (e.g., a knowledge graph that represents knowledge about certain medical concepts), and can be synced across devices and restored from a cloud-based service provider (e.g., when a user moves to a new user device). In essence, UDCs create a stable point around which additional enhancements to the organization and presentation of health data can be added.

Turning now to a particular example, in this example, a user device, including a particular application, may download health data from one or more electronic health record (EHR) systems. This health data may represent labs, medications, notes, diagnosis, allergies, imaging, and the like as generated by one or more health care professionals that have treated a user of the user device (e.g., a patient). As patients often see health care professionals at different facilities that may or may not share an EHR system, the user device may be configured to process health data from different EHR systems, each of which may store the health data slightly differently (e.g., because they have adopted certain health-data standards differently). The user device may download a reference ontology from a service provider and store the reference ontology in a first storage location on the user device. The reference ontology may include knowledge about medical concepts and can be used to enhance health data stored on the user device. Once the health data has been downloaded, the user device may process the health data (e.g., download, index, organize, etc.) and store the health data in a second storage location of the user device. Part of processing the health data may include generating a personalized data graph that represents the specific health data. Generating this data graph may include identifying medical concepts present in the health data using a coding technique, generating nodes of the personalized data graph based on the concepts, enhancing existing nodes, and/or creating new nodes based on associations between medical concepts and the knowledge graph. The user may also utilize the application to view the health data, as represented by the personalized data graph, and generate UDCs to represent customizations of the health data. For example, the application may be used to mark a certain health record as a “favorite” for quick access (e.g., a lab result), to add records to a list (e.g., a list of all active medications), to give a record a nickname (e.g., give record of “Omeprazole” the nickname of “heartburn medicine”), and to perform other customizations as described herein. The UDCs may be generated as nodes in the personalized data graph. Relationships between UDC nodes and other UDC nodes and UDC nodes and other non-UDC nodes may be stored by the nodes. Because the UDC nodes are part of the personalized data graph, the UDC nodes may be stored in the second storage location.

The examples described herein address a number of technical problems and provide for a number of technical improvements. In some examples, these improvements additionally improve the functioning of various components of a system in which the techniques are implemented. The techniques described herein provide for customization of health data, while also reducing on-device storage requirements and bandwidth needed for data transfer. This is because user customization data is separated from the reference ontology data. This allows a single reference ontology between any number of users who store data on the same device. For example, multiple profiles (e.g., parent and children) representing different people's health data can share a single reference ontology. Thus, unlike conventional systems that might store a reference ontology for each profile, storage savings are achieved and bandwidth conserved because only one ontology is stored, received, and updated (e.g., periodically). Additional storage and bandwidth savings are realized at other devices that collect health data and are connected to the user devices, such as smartwatches and other wearable devices. Because UDCs can be synced and shared to these other devices that do not have their own reference ontologies, these other devices do not need to take on that bandwidth storage cost.

The techniques described herein provide for customization of health data, while also improving memory utilization, e.g., by providing for more efficient searching of health data. For example, UDCs may allow for more efficient searching and retrieval of medical records (user data) and/or medical concepts (reference data) than conventional systems. This may be because such searching and retrieval may be performed by predicates based on relationships between the user data and reference data (e.g., “groups by”, or “is elements of a list”:). Conventional systems may require the user device to enumerate all the medical records, looking for their related medical concepts, holding both in memory, until all the records for a given concept had been found. As can be seen, the predicate based searching may not include the same memory-intensive requirements.

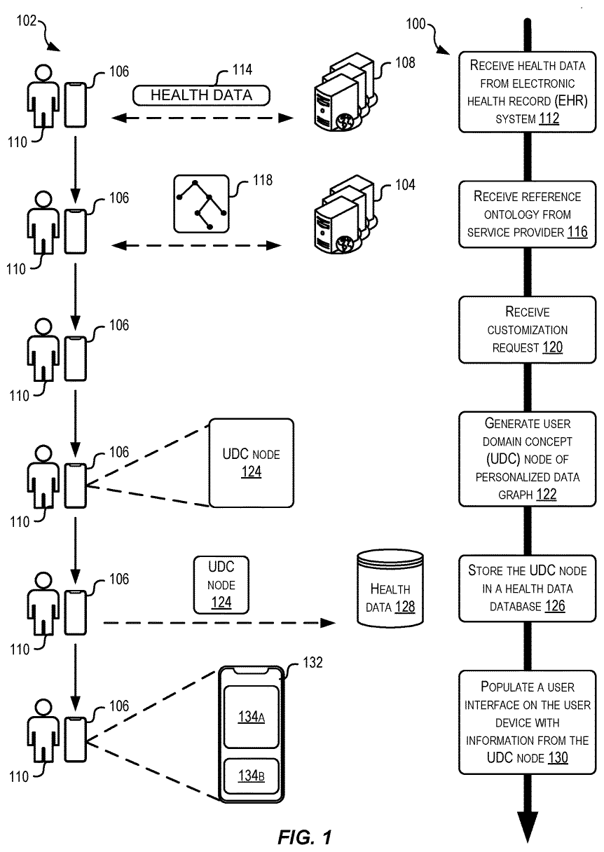

Turning now to the figures, FIG. 1 illustrates a block diagram 102 and a flowchart showing a process 100 for generating user domain concept nodes of personalized data graphs, according to at least one example. The diagram 102 includes a service provider 104, which is any suitable combination of computing devices such as one or more server computers, which may include virtual resources, capable of performing the functions described with respect to the service provider 104. For example, the service provider 104 may include one or more different servers and/or services dedicated to handling different aspects of enabling user devices to connect with EHR systems, maintain reference ontologies, or share reference ontologies with user devices.

The diagram 102 also includes a user device 106. The user device 106, which may be any suitable device such as a smartphone, tablet, smartwatch, wearable device, laptop computer, or desktop computer, may be configured to interact with the service provider 104 and an electronic health record (EHR) system 108. For example, the user device 106 may include one or more network radios to enable network connections with remote systems such as the service provider 104 and the EHR system 108. In some examples, the user device 106 may include one or more applications, which may include custom-built algorithms and other logic, to enable performance of at least some of the techniques described herein. The user device 106 may also include storage media for storing computer-executable instructions (e.g., that make up the application) and other data such as described herein. The user device 106 may be operated by a user 110. The service provider 104 and/or the EHR system 108 may maintain profiles for the user 110. For example, the service provider 104 may maintain a user profile relating to digital services offered by the service provider (e.g., messaging, cloud backup, music streaming), and the EHR system 108 may maintain a user profile relating to medical services offered by a health institution associated with the EHR system 108. The user profile at the EHR system 108 may be a patient profile as the user 110 may be a patient of the health institution.

The diagram 102 also includes the EHR system 108. The EHR system 108 is associated with at least one health institution and used to manage electronic health record data from the institution. In some examples, a single EHR system 108 is associated with multiple health institutions and used to manage electronic health record data from the multiple institutions. In particular, the EHR system 108 may store, organize, and/or otherwise manage health record data generated by medical professionals of the health institution. The EHR system 108 may include one or more gateways, each including one or more endpoints to enable multiple connections between the EHR system 108 and other electronic devices. In some examples, user devices, such as the user device 106, may interact with the EHR system 108 using any suitable interfaces such as gateway application programming interfaces (API) or via patient portals including graphical user interfaces. The gateway APIs may define a set of function calls for communications between the EHR system 108 and the user device.

FIGS. 1, 5, and 6 illustrate example flow diagrams showing processes 100, 500, and 600, according to at least a few examples. These processes, and any other processes described herein, are illustrated as logical flow diagrams, each operation of which represents a sequence of operations that can be implemented in hardware, computer instructions, or a combination thereof. In the context of computer instructions, the operations may represent computer-executable instructions stored on one or more non-transitory computer-readable storage media that, when executed by one or more processors, perform the recited operations. Generally, computer-executable instructions include routines, programs, objects, components, data structures, and the like that perform particular functions or implement particular data types. The order in which the operations are described is not intended to be construed as a limitation, and any number of the described operations can be combined in any order and/or in parallel to implement the processes.

Additionally, some, any, or all of the processes described herein may be performed under the control of one or more computer systems configured with specific executable instructions and may be implemented as code (e.g., executable instructions, one or more computer programs, or one or more applications) executing collectively on one or more processors, by hardware, or combinations thereof. As noted above, the code may be stored on a non-transitory computer-readable storage medium, for example, in the form of a computer program including a plurality of instructions executable by one or more processors.

The process 100 begins at block 112 by the user device 106 receiving health data 114 from one or more EHR systems 108. As part of receiving the health data 114 at block 112, the user device 106 and the EHR system 108 may perform a credential validation procedure. For example, the EHR system 108 may make an endpoint of the system available via a web-based portal, and the user 110 may use the user device 106 to sign into the web-based portal using a set of credentials (e.g., username/password, patient identifier). Upon validation of the credentials, the service provider 104 may send the requested health data 114, which may include establishing a persistent data connection with the user device 106. In this manner, as data is updated by the EHR system 108 (e.g., files are added to a patient record), the EHR system 108 may automatically (e.g., at some predefined cadence or responsive to certain triggers) send the new health data to the user device 106. As described herein, the user device 106 may establish and simultaneously maintain multiple such persistent data connections with multiple different EHR systems 108 associated with different health institutions (e.g., a local clinic, a regional private hospital chain, an urgent care facility, and a university-affiliated emergency room).

While not explicitly shown in FIG. 1, the process 100 may also include the user device 106 processing the health data 114. This may include indexing, organizing, and otherwise adjusting the health data 114 to improve the presentation of the health data 114. This may include generating a personalized data graph that represents medical concepts identified from the health data 114. The personalized data graph, as described elsewhere herein, may be a relational data structure that is built by the user device 106 using the health data and, in some examples, other data. The personalized data graph represents a representation of the user's 110 health data 114 that is unique, because the user's health data 114 is unique. In some examples, the personalized data graph may be further customized based on the reference ontology and the UDCs described herein.

At block 116, the process 100 includes the user device 106 receiving a reference ontology 118 from the service provider 104. The reference ontology 118 may be shared as part of an update, which may be in-band or out-of-band. In some examples, the reference ontology 118 may be pushed to the user device 106 by the service provider 104 whenever new information has been added to the reference ontology 118. The reference ontology 118 may include any suitable collection of health related information organized based on some predefined manner. For example, the reference ontology 118 may be organized by medical concepts, medical codes, medical terms, and/or any other suitable organizational structure. In some examples, the reference ontology 118 may include a collection of information that can be used to enhance or otherwise increase the richness of the health data obtained at 112. For example, information from the reference ontology 118 may be used to provide additional information about a cancer diagnosis present in the health data 114. In this example, the additional information may include articles, multimedia content, websites, and the like for learning more about the particular diagnosis. This additional information from the reference ontology 118 can be linked from the reference ontology and stored in the personalized data graph, as described herein.

At block 120, the process 100 includes the user device 106 receiving a customization request. In some examples, the user 110 may use a user interface of the user device 106 to input the customization request (e.g., a command received via a touch interface of the user device 106). The user interface may be presented by an application running on the user device 106, which may be in communication with the service provider 104. The customization request may include a request to customize some aspect of the health data. For example, this may include marking a record of the health data as a favorite, adding a record of the health data to a list, giving a record a nickname, associating two health records (not previously associated), and/or performing other similar operations.

At block 122, the process 100 includes the user device 106 generating a user domain concept (UDC) node 124 of the personalized data graph. This may be based on the customization request received at block 120. Generating the UDC node 124 may include executing logic based on the customization request. For example, depending on what type of customization was requested at block 120, the user device 106 may generate aspects of the UDC node 124 differently. In some examples, the UDC node 124 may include relationship information that indicates its relationship, if present, to other UDC nodes and/or other nodes of the personalized data graph. As described herein, the UDC node 124 may be added to and saved in connection with the personalized data graph.

At block 126, the process 100 includes the user device 106 storing the UDC node 124 in a health data database 128. The health data database 128 may be used to store other health data such as the health data 114, health data generated at the user device 106 and/or received from an associated user device (e.g., a smartwatch, step counter, wearable sensor). Storing the UDC node 124 (and the personalized data graph) in the same location as the health data ensures that the UDC nodes 124 will persist so long as the health data database 128 persists. The data in the health data database 128 is not updated by the service provider 104, thus, the UDC nodes 124 may remain unchanged by the service provider 104. In some examples, the health data database 128 may be synced with a cloud-based version of the database stored by the service provider 104. In this manner, the data from the health data database 128 may be synced with other user devices 106 (e.g., devices associated with a user profile belonging to the user 110).

At block 130, the process 100 includes the user device 106 populating a user interface 132 of the user device with information from the UDC node 124. This action at block 130 may be performed responsive to a request, such as a request from the user 110 received at the user interface 132 of the user device 106. For example, if the UDC node 124 represented a “favorited” record and the request at block 130 was to view favorited records, populating the user interface 132 may include identifying which UDC node(s) 124 represent favorited records, accessing information from those node(s) 124, and using the accessed information to populate the user interface 132. In some examples, the user interface 132 may include at least two areas 134a and 134b. In some examples, populating the user interface 132 at block 130 may include populating at least one area 134. In some examples, other area(s) (e.g., 134c-N) may be populated with information obtained from other nodes of the personalized data graph and/or from the reference ontology 118.

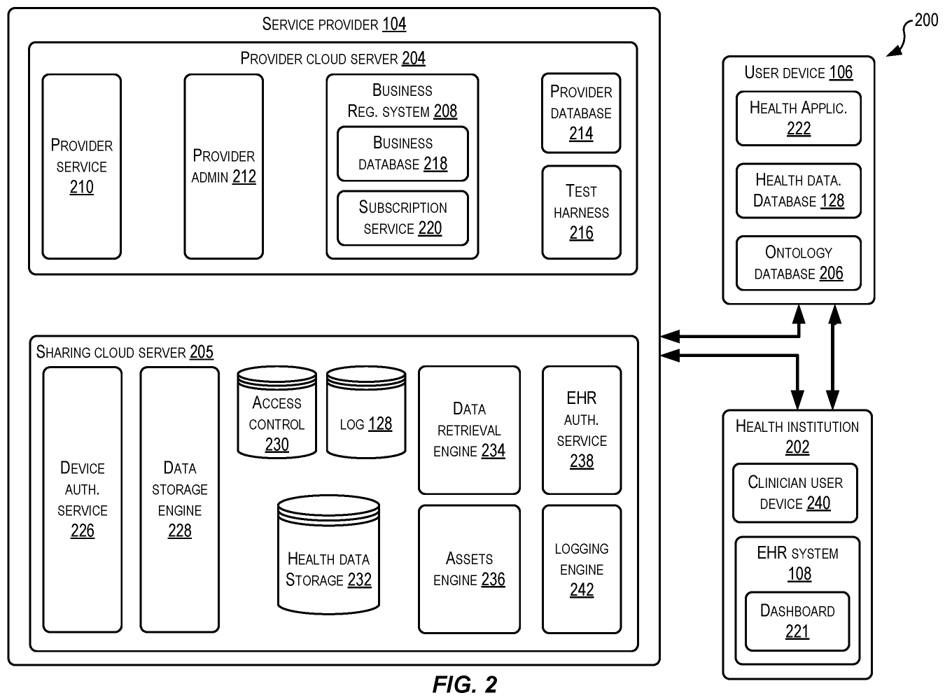

FIG. 2 illustrates a block diagram showing an example architecture or system 200 for enabling techniques relating to generating user domain concept nodes of personalized data graphs, according to at least one example. The system 200 includes a few elements introduced in FIG. 1. In particular, the system 200 includes the service provider 104, the EHR system 108 (e.g., one or any other suitable number of EHR systems) associated with a health institution 202, the user device 106, and the health data database 128. As appropriate and as illustrated by the arrows, the elements of the system 200 may be communicatively coupled via one or more suitable communication networks. For example, the service provider 104, the user device 106, and the EHR system 108 may be configured to communicate with each other via one or more networks, as described herein and is known in the art.

Beginning with the service provider 104, the service provider 104 includes a provider cloud server 204 and a sharing cloud server 205. Generally, the provider cloud server 204 includes one or more server computers, which may be virtual, and is configured to onboard health institutions to enable sharing of health data, manage registrations of the health institutions once onboarded, and conduct tests of endpoints of the EHR systems 108 of the health institutions 202 to ensure correct operation with the sharing system provided by the service provider 104. Generally, the sharing cloud server 205 includes one or more server computers, which may be virtual, and is configured to manage sharing of health data by user devices 106 with the service provider 104 and the health institution 202. In particular, different elements of the sharing cloud server 205 manage different aspects of sharing including authorization of user devices 106 and EHR systems 108, data retrieval, and the like.

The provider cloud server 204 includes a business registration system 208, a provider service 210, a provider admin 212, a provider database 214, and a test harness 216. Business registration system 208 may be any suitable collection of hardware and software components configured to collect, store, update, and otherwise manage business locations including those of the health institutions 202. For example, the business registration system 208 may include a business database 218 and a subscription service 220 to enable subscription of the EHR systems 108 of the health institutions 202. When a health institution 202 is subscribed and active, the user devices 106 may be allowed to share health data with the EHR system 108 and connect to and download health record data from the EHR system 108 (e.g., a gateway of the EHR system 108).

As part of subscribing and managing subscriptions, the subscription service 220 may include functionality to collect, store, update, and otherwise manage business locations. In some examples, the subscription service 220 provides one or more user interfaces by which authorized users of the health institution 202 may input information about their location. This information may include geographic information (e.g., a physical address and pin on a map), image information (e.g., logos), contact information (e.g., business, legal, technical), and any other information relating to a business. The subscription service 220 may also be configured to create and/or update record entries in the business database 218 based on information received. For example, an authorized user associated with the health institution 202 may share business information with the subscription service 220. Once this information has been shared and validated, the business information may published for public consumption (e.g., indexed for searching, made available on a map platform, shared directly with users).

The business database 218 may maintain the collected business information and any relationships between entities represented by the business information. In some examples, the business registration system 208 may be used to register other business entities (not just health institutions 202). Records for the health institutions 202 may be maintained in both the business database 218 and a provider database 214 maintained by the provider cloud server system 204. In some examples, the business registration system 208 shares business information with the sharing cloud server 205 in any suitable manner.

Turning now to the provider service 210, generally, the provider service 210 may validate the EHR systems 108, maintain information about the health institutions 202 and associated EHR systems 108, enable searching of the health institutions 202 associated with the EHR systems 108, and manage access of the user devices 106 to the EHR systems 108. The provider cloud server 204 may be implemented in the “cloud.” The provider service 210 may also maintain information about the health institutions 202 and associated EHR systems 108 in the provider database 214, enable searching of the health institutions 202 associated with the EHR systems 108, and manage access of the user devices 106 to the EHR systems 108. In some examples, the user device 106 sends requests to search for the health institutions 202 to the provider service 210. The provider service 210 processes these requests and returns results. In some examples, as part of establishing a connection with one of the EHR systems 108, the user device 106 will check in with the provider service 210 to determine whether any configuration information associated with the EHR system 108 has changed. The configuration information, which may be stored in the provider database 214, may include API information, provider identifiers, status indicator information, and any other suitable information relating to the configuration of the EHR system 108 and/or other entities associated with the EHR system 108.

As part of subscribing a health institution 202, the test harness 216 may be used to test and/or otherwise validate that a test user using a test user device 106 can connect to and download data from the EHR system 108. In this manner, the test harness 216 may simulate actions that one of the user devices 106 may perform to connect to the EHR system 108. In some examples, the test harness 216 may test this connection when a health institution 202 is first subscribed and at other times after the initial subscription. For example, the connection may be tested periodically when certain conditions are fulfilled and under any other circumstance. If the test is positive, a status indicator(s) in the provider database 214 associated with the health institution 202, the EHR system 108, and/or a gateway associated with the EHR system 108 may be updated to reflect that the EHR system 108 or the gateway is/are active. If the test is negative, the status indicator(s) may be updated to reflect that the EHR system 108 is inactive. When active, the user devices 106 may be capable of connecting to the gateways of the EHR systems 108. When inactive, the user devices 106 may be unable to connect to the gateways of the EHR systems 108.

The provider admin 212 may generally be used by an administrator or other authorized user to manage aspects of the provider cloud server 204.

The user device 106 may include a health application 222, the health data database 128, and an ontology database 206. Generally, the user devices 106 may be associated with and operated by different users (e.g., patients of the health institutions 202). Functionally, the health application 222 may enable the user devices 106 to communicate with the provider cloud server 204 (e.g., to search for the health institutions 202, obtain configuration information about the health institutions 202, and perform other techniques), communicate with the EHR systems 108 of the health institutions 202 (e.g., to download reference ontologies, to download data packages including electronic health records and/or updates to electronic health records and to perform other such techniques), and communicate with the sharing cloud server 205 to upload encrypted health data, and to perform the techniques described herein for generating UDCs relating to a personalized data graph, and perform other techniques described herein.

The sharing cloud server 205 may include a device authentication service 226, a data storage engine 228, an access control storage 230, a health data storage 232, a data retrieval engine 234, an assets engine 236, an EHR authentication service 238, and a logging engine 242. Generally, the device authentication service 226 is used to authenticate users of the user devices 106 for accessing the service provider 104 and/or the EHR system 108. For example, the device authentication service 226 may use a two-way authentication or mutual authentication such as internet key exchange (IKE), secure shell (SSH), or transport layer security (TLS). In an example that uses TLS, a client-side X.509 certificate may be used to authenticate the identity of the client (e.g., a user device 106) to the server (e.g., the sharing cloud sever 205), and an X.509 certificate may be used to authenticate the server to the client. In some examples, the mutual authentication may include the use of usernames and passwords in addition to or instead of certificates. In some examples, the access control storage 230 may store credential data usable by the device authentication service 226.

The data storage engine 228 may be configured to receive encrypted health data from the user device 106 and store it in the health data storage 232. For example, the data storage engine 228 may include a set of method calls to communicate with the user device 106 and store the encrypted health data. The encrypted health data may be passed to the health data storage 232 using a Blob application programming interface (API) to push data to the health data storage.

The access control storage 230 generally stores credentials for health institutions and/or providers. Information in the access control storage 230 may be accessed by the provider admin 212 as part of when an EHR system 108 makes a request for health data storage 232.

The health data storage 232 may be used to store encrypted health data obtained from the user device 106. The data retrieval engine 234 may be used to retrieve the encrypted health data from the health data storage 232 responsive to requests from a clinician user device 240 (e.g., an example of the user device 106) providing a dashboard 221. For example, the data retrieval engine 234 may use a Blob API to pull data from the health data storage 232.

The assets engine 236 is configured to manage assets of the sharing cloud server 205. This may include removing or otherwise deleting old or stale data in the health data storage 232. In some examples, such deletion may be part of a garbage collection routine or may be requested or otherwise instructed by the user device 106. For example, the user device may change the way the health data is stored, and the assets engine 236 may make the adjustments in the health data storage 232.

The EHR authentication service 238 is used to authenticate the clinician user device 240 and/or the dashboard 221. For example, the EHR authentication service 238 may use a decentralized authentication protocol such as OpenID to authenticate the clinician user device 240 and/or the dashboard 221.

The clinician user device 240 may be operated by a clinician or other authorized user to access the health data of the user. The dashboard 221, which may be presented by the clinician user device 240, may be an application such as web application accessible via a web browser of the clinician user device 240 or any other suitable application accessible by the clinician user device 240. In some examples, the dashboard 221 may be hosted by the EHR system 108 and may be the typical dashboard 221 used by the clinician to access electronic health records stored by the EHR system 108. To enable the dashboard 221 to view the health data from the service provider 104, as described herein, the dashboard 221 may be slightly modified to include a link, icon, or other graphical element to enable the clinician to see the health data loaded into the EHR using the techniques described herein. In some examples, EHR system 108 communicates with the health application 222 to authenticate the user of the user device 106. Such communications may occur using an existing Health Level Seven International Standard (HL7) such as Fast Healthcare Interoperability Resource (FHIR) standard describing data elements, data formats, and APIs for exchanging electronic health records. In some examples, the open source implementation of the FHIR standard, referred to as SMART on FHIR (Substitute Medical Applications, Reusable Technologies on FHIR), may be appropriate.

The user device 106 may include the health application 222 and the health data database 128. As described herein, the health application 222 may be used to perform the techniques relating to generating UDC nodes, storing health data using such nodes, and the like. The health data database 128 may be used to store the health data that is shared, as described herein, and any other health record data.

Logging engine 242 may be used to log transaction information related to requests for health care data received from the clinician dashboard 221 and/or the clinician user device 240, as described in further detail herein. In some examples, the logging engine 242 may also include encryption and/or decryption algorithms for performing cryptographic functions, as described herein. The logging engine 242 may store transaction information in the health data database 128, as introduced herein.

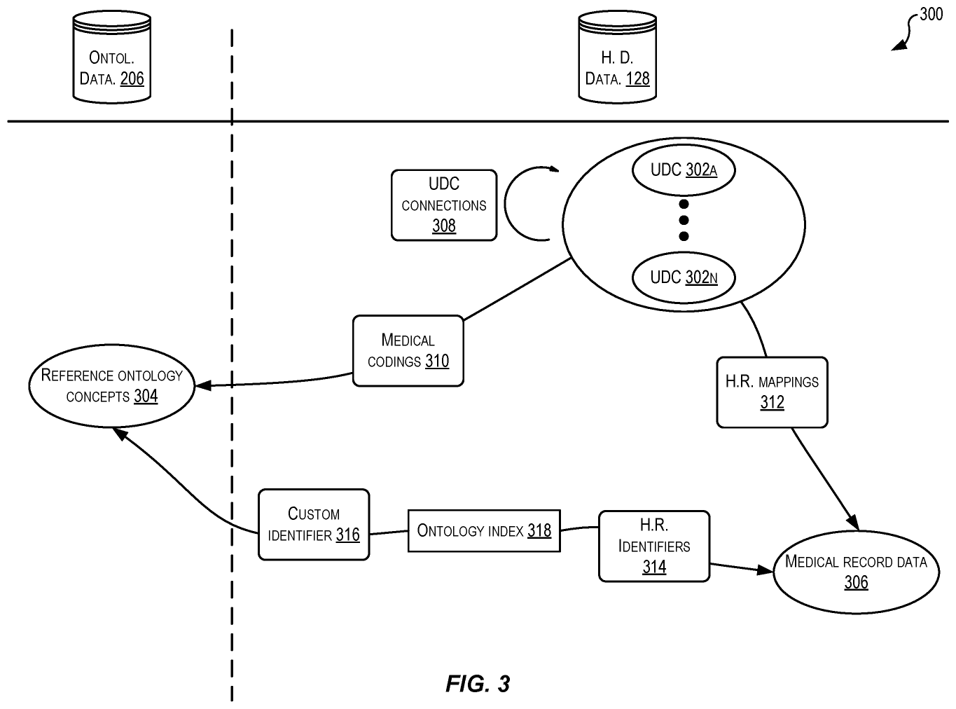

FIG. 3 illustrates a diagram 300 showing example user configurations for storing health data on a user device relating to user domain concept nodes of a personalized data graph, according to at least one example. The diagram 300 includes the health data database 128 and the ontology database 206, both of which may be stored by the user device 106 as described herein. In the diagram, the vertical dashed line depicts the demarcation between what data is stored in each of the two databases 128 and 206. For example, the diagram 300 includes one or more UDCs 302a-302N, reference ontology concepts 304 (e.g., knowledge graph), and medical record data 306 (e.g., samples of health records) and identifies where these different information elements are stored, e.g., the reference ontology concepts 304 in the ontology database 206 and the UDCs 302 and the medical record data 306 in the health data database 128. The diagram 300 also depicts relationships between the UDCs 302, the reference ontology concepts 304, and the medical record data 306.

Beginning with the UDCs 302, any suitable number of UDCs 302 may be included, depending on the level of customization desired. A single UDC 302 (e.g., the UDC 302a) may store a connection 308 with a different UDC 302 (e.g., the UDC 302N). The connections 308 may be one to one (e.g., one UDC to one UDC) or one to many (e.g., one UDC to many UDCs). In some examples, information about the connections 308 is stored by the UDCs 302. This may include information that uniquely identifies the UDC 302, identifies the relationship, and the like. Because the information about the connections 308 between UDCs is stored in the health data database 128, this information will remain on the user device, even if the reference ontology concepts 304 change. Depending on the use case and the customization request received by the user device, the user device may generate UDC connections 308 between UDCs 302 and update the relevant UDCs 302 with information descriptive of the connections 308.

The UDCs 302 may also be connected to, based on, or otherwise associated with the reference ontology concepts 304 via medical codings 310. The UDC 302 may store information about the medical codings 310, which in turn may reference a particular node of the reference ontology concepts 304. The medical codings 310 may be numerical or other codes that represent medical concepts present in the reference ontology concepts 304. For example, the medical codings 310 may correspond to an industry standard for medical coding such as International Classification of Diseases, 10^{th} Edition, Clinically Modified (ICD-10-CM), Current Procedure Terminology (CPT), ICD-10-Procedural Coding System, National Drug Codes (NDC), and any other suitable coding standard. Depending on the use case and the customization request received by the user device, the user device may generate links with the reference ontology concepts 304 by updating the UDCs 302 with information descriptive of the medical codings 310 by which the UDCs 302 are linked with the reference ontology concepts 304.

The UDCs 302 may also be connected to, based on, or otherwise associated with the medical record data 306 via health record mappings 312. The UDCs 302 may store information about the health record mappings 312, which reference specific medical record data 306. For example, a health record mapping 312 may represent an association between a prescription present in the medical record data 306 and a UDC 302 that represents that the prescription has been favorited by a user. The UDC 302 may represent any suitable aspect of the medical record data 306, as represented by the health record mappings 312. Depending on the use case and the customization request received by the user device, the user device may generate links with the health record data 306 by updating the UDCs 302 with information descriptive of the health record mappings 312 by which the UDCs 302 are linked with the health record data 306.

The medical record data 306 may be connected to or otherwise associated with the reference ontology concepts 304. Individual medical records represented by the medical record data 306 may be represented by health record identifiers 314. The health record identifier 314, in some examples, may be a universally unique identifier (UUID) or other suitable identifier capable of uniquely identifying the medical record data 306. The health record identifiers 314 may be generated by the user device after or as the user device receives the health record data 306 (e.g., from an EHR system). Individual reference ontology concepts 304 may be represented by custom identifiers 316. The ontology index 318 may be used to map between the health record identifiers 314 and the custom identifiers 316.

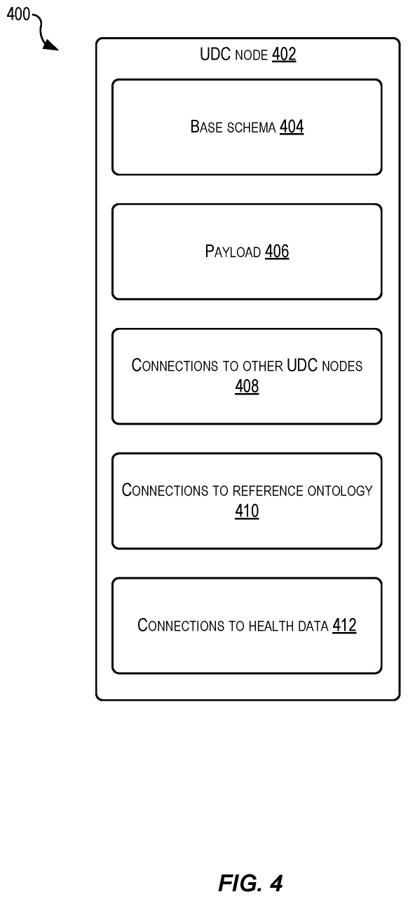

FIG. 4 illustrates a block diagram showing an example data model 400 for generating user domain concept nodes of personalized data graphs, according to at least one example. The data model 400 represents an example UDC node 402 and corresponds to the diagram 300. The UDC node 402 can be modeled as having a base schema 404, a payload 406, connections to other UDC nodes 408, connections to reference ontology 410, connections to health data 412, and any other suitable element. The UDC node 402 may be included in a personalized data graph (e.g., a table that includes UDC nodes and a representation of the health data of the user). The health data may be downloaded from EHR systems, generated by the user device, obtained from a sensor of the user device or a peripheral device, and/or entered by a user of the user device.

Beginning with the base schema 404, the base schema 404 may include the following fields: a user domain concept identifier (udc_id) (e.g., a unique identifier for the UDC node, which may also correspond to a row identifier (row_id)), universal unique identifier (uuid) (e.g., an identifier that uniquely identifies the UDC node), schema (e.g., a nullable string that identifies the schema), type (e.g., an enum data type that distinguishes the UDC node from other types of data nodes), deleted (e.g., an “bool” data type relating to a deleted state), creation date (e.g., information that identifies the date that the UDC node was created), modification date (e.g., information that identifies the most recent modification of the UDC node), os_version (e.g., information that identifies the version of the operating system), build (e.g., information that identifies the build of the operating system), synch anchor (e.g., information that allows tracking of which changes have not yet been pushed/pulled to/from various sync endpoints), and sync provenance (e.g., information that identifies sync origins).

In order to avoid some sync ordering issues and a large increase in the number of new sync entities, in some examples, the UDCs nodes may share a single common sync entity. In this example, the UDC type specific payload data may be serialized based on the type.

Sync anchors may allow tracking which changes have not yet been pushed/pulled to/from various sync endpoints. The sync anchors may be represented in the database as an integer column in the base table for a given entity. For most entities that column may simply be the auto-incrementing rowid. In some examples, the sync anchor may increase as rows are inserted, or deleted and re-added if a “modification” is required to any row is performed.

In order to maintain device-local references between the UDC rows in various tables, the sync_anchor column may be used, that is incremented when the UDC is updated. In some examples, the default conflict resolution strategy for a UDC (unique by UUID) may be “last modification wins.” This strategy may be overridden for a particular UDC type. In some examples, the UDC type identifier may be a tuple consisting of (plugin schema identifier, type enum). This may enable integration of UDCs generation using plug ins.

Turning now to the payload 406, the payload 406 may be different depending on the UDC type. Thus, each UDC type may include custom data models, persistence (database tables), and serialization support specific to its function and purpose. For example, connections to health data 412 and connections to other UDCs nodes 408 may be implemented by the base entity. As an example, a payload for a medication example may include:
A Consumer Friendly Name (from referenced ontology)
A Nickname (user entered string)
An Icon (user selected enum)
A Status (active/inactive)
   To allow for backwards/forwards compatible sync of UDCs between devices with different operating systems and reference ontology versions, UDCs may store mutable properties in a UDC property collection. This collection may be made up of generic, versioned UDC property objects. These collections of versioned properties can be merged or diffed in such a way to keep only the latest version of a given property, while not losing properties that may be unknown (because they come from a future operating system). For example, if a future operating system version adds a property “user selected nickname” to a UDC, this property may sync back to devices running older operating system versions that do not have this property type defined and will be preserved even if other properties in the collection are modified/updated on the older device.

Turning now to the connections to other UDC nodes 408, the connections to other UDC nodes 408 may be persisted with a schema including: rowid, udc_id (foreign key referencing this UDC), connection_type (enum), target_uuid (referencing UDC UUIC of other “connected” UDC). Changes (adding or removing a connection for a UDC) may change the UDC's sync anchor, so that the UDC may sync with its current set of connections. In some examples, the target_uuid is used rather than target_udc_id. This may be helpful to support multi-device sync cases where a UDC and its connections appear on a device before all UDCs have connected to it.

Turning now to the connections to reference ontology 410, the connections to reference ontology 410 may be configured to enable efficient searching of connected reference ontology node(s) given a UDC node and to maintain connections after restore from backup or sync to a new device. In some examples, the UDCs will persist/sync the codings (e.g., medical codings 310) for the connected ontology nodes. These codings may come from the health record the user interacted with to create the UDC, or they could be pulled from the reference ontology during manual creation of the UDC. An example schema for these connections may include: row_id, udc_id (foreign key referencing this UDC), system, code, version, display_string. In some examples, system, code, version, and display_string may be nullable foreign key references to rows in a user domain concept coding strings table that exists to deduplicate storage of common coding strings. In some examples, udc_id, system, code, version may form a unique tuple. The user domain concept coding strings may include row_id, string (non-null unique string). In some examples, storing the system/codes for a UDC may avoid the need to build an index table mapping UDCs to reference ontology nodes, since these mappings can be determined by efficient run-time queries.

Turning now to the connections to health data 412, the connections to health data 412 may be configured to enable one or more of the following: efficiently find connected health record data (e.g., samples) given UDC, efficiently find connected UDC given sample(s), handle connections to very large numbers of samples (e.g., a UDC connected with all heart rate samples), maintain or rebuild connections after restore from backup or sync to new device, function without a reference ontology, handle new medical record data represented by new UUIDs, handle change in Fast Healthcare Interoperability Resources (FHIR) identifiers (e.g., DSTU-2 to R4 conversion) for medical records, determine in-memory if a given sample is connected to a UDC (e.g., to support change detectors).

In some examples, each UDC may persist and sync a set of “connected sample descriptions.” Any sample that matches one of these descriptions may be considered connected to the UDC. For example, a UDC that is connected to all samples with heart rate type identifier. The connections to health data 412 may be useful for efficient bi-directional queries (e.g., samples connected to a UDC, and UDCs to which a sample is connected) if the persisted connection has a structured form in the database rather than being a serialized blob. This may allow for the direct creation of sqlite predicates that can find UDCs for a given sample and vice versa. For example, properties to represent a given sample connection description may be added as each new type of sample connection is proposed for some specific use case. The result may be a highly denormalized table, with nullable columns representing connection description specific properties.

An example schema for the connections 412 may include rowid, udc_id (foreign key referencing this UDC), data_type (nullable object type to which the connection applies), quantity (nullable health data Quantity). In some examples, certain connections may be difficult to query directly from a predicate because these connections are represented by serialized blobs in the database. In order to support querying these types of connections, the UDC node may maintain a persisted device-local UDC to sample mapping for indexed lookups in the UDC mapping entity. This mapping may be built and maintained while UDCs are generated and refreshed. The samples to be indexed may be defined in the “connected sample predicate.”

Manually entered medical record samples may not have any reference coding (e.g., if they are entered for a UDC that itself is not connected to a reference ontology concept). In order to maintain the connection between the record and UDC, the manually entered record may be given some additional metadata at creation time that maps to the UDC. This may maintain the connection even if later on the UDC and/or record begin mapping to reference ontology concepts.

In some examples, the UDC node 402 may also enable notification of user devices when UDCs are added or removed, notification of user devices when UDCs are modified, notification of user devices when new health records are connected to a UDC, and notification of user devices when new health records are added that are not connected to any UDC (e.g., so that the user can be prompted to add a UDC connection to the health record sample).

In some examples, the UDC node 402 may include relationship information that identifies, for the specific node, a relationship with respect to other nodes (e.g., UDC nodes or concept nodes). For example, such relationship information may include things such as a list member (e.g., defines that the node is a member of a list), can support (e.g., defines that the node can support), child (e.g., defines that the node is a child of a different node), parent (e.g., defines that the node is a parent of a different node), more specific (e.g., defines that the node is more specific than the other node), or less specific (e.g., defines that the node is less specific than the other node).

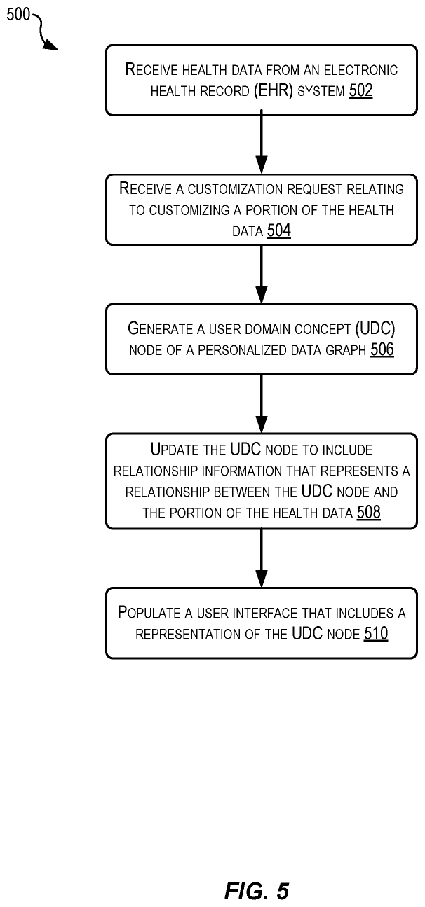

FIG. 5 illustrates a flow chart showing an example process 500 for generating user domain concept nodes of personalized data graphs, according to at least one example. The process 500 may be performed by the health application 222 (FIG. 2) of the user device 106 (FIG. 1). In some examples, at least some portion (or the entirety thereof) may be performed by the service provider 104 (FIG. 1).

The process 500 begins at block 502 by the user device 106 receiving health data from an electronic health record (EHR) system. The health data may be associated with a patient profile maintained by the EHR system. A user of the user device 106 may use the user device 106 to log into a system maintained by the EHR system to have the system validate that the user is authorized to access the health data (e.g., the user is the same user identified in the patient profile or is otherwise associated with the patient profile). Thus, as part of receiving the health data from the EHR system, the user device 106 may send a login request to the EHR system, which may include credentials of the user of the user device 106.

The health data may include clinical health record data, sensor data, and/or any other suitable type of health data. In some examples, the clinical health record data is received from the electronic health record system or from a different health record system associated with a different health institution. In some examples, the sensor data is generated by a sensor of the mobile user device or received from a different user device (e.g., accessory device such as a watch, wearable monitor) associated with the user device. In some examples, a first portion of the health data is obtained by the user device from the electronic health record system, and a second portion of the health data is obtained by the user device from a different electronic health record system associated with a different health institution. In some examples, the health data is organized into a plurality of categories. The health data may be associated with a user profile of the user device or a user profile of a member of a predefined group (e.g., a device family).

At block 504, the process 500 includes the user device 106 receiving a customization request relating to customizing a portion of the health data. The customization request may be received from the user of the user device or triggered in an automated manner. For example, the health application, which may be useable by the user to view and organize the health, may also include functionality to enable certain customizations. Such customizations may include, for example, adding health records to a list, favoriting health records, grouping health records, and the like. In this example, the user may use a user interface at the user device 106 to interact with the health application to customize the health data, which in turn may be received and processed by the user device 106 as a customization request. In some examples, the customization request may include at least one of a request to favorite the portion of the health data, a request to give the portion of the health data a nickname, a request to add the portion of the health data to a list, a request to combine the health data with other health data, a request to connect an existing UDC node with one or more other UDC nodes of a personalized data graph.

In some examples, block 502 may be omitted, and the process 500 may begin at 504 by the user device 106 receiving the customization request. In this example, however, the customization request may relate to generating a UDC node that is not associated with health data and/or concept nodes. For example, a user may generate an entirely new UDC node that is independent of health data and/or concept nodes. In some examples, such a UDC node may also be generated when a user inputs health data at the user device 106, rather than the user device 106 receiving the health data from the EHR system. For example, the user device 106 may enable the user to enter information about a health-related event, condition, or diagnosis. This information may be pushed out and saved as a health record and, in some examples, may also be used to create a UDC node to capture any additional customization of the event, condition, or diagnosis.

At block 506, the process 500 includes the user device 106 generating a user domain concept (UDC) node of a personalized data graph. Generating the UDC node may include generating based at least in part on the customization request. The UDC node may include customized information that represents the customized portion of the health data. In some examples, the UDC node may include at least some of the information described with respect to the UDC node 402 of FIG. 4. For example, the customization request may associate the UDC node with the portion of the health data, a concept of a knowledge graph, another UDC node, or any other suitable data element.

In some examples, the portion of the health data may include a plurality of health data samples, and the UDC node may include individual connections with each of the plurality of health data samples. For example, the plurality of health samples may be a plurality of heart rate measurements taken at different times, and possibly even pulled from different health records, and the UDC node may represent associations between these different heart rate measurements.

At block 508, the process 500 includes the user device 106 updating the UDC node to include relationship information that represents a relationship between the UDC node and the portion of the health data. In some examples, updating may be based at least in part on generating the UDC node. In some examples, generating and updating may be performed as a single operation, as separate operations, and/or as an interrelated operation (e.g., the node may be generated then updated to include information that populates fields of the node).

In some examples, the process 500 may further include the user device 106, after receiving the health data, indexing the health data to assign reference coding to individual portions of the health data. In this example, updating the UDC node to include the relationship information may include updating the UDC node to include a reference coding assigned to the portion of the health data.

At block 510, the process 500 includes the user device 106 populating a user interface that includes a representation of the UDC node. In some examples, the user interface may be presented within an application such as the health application. The user interface may include one or more areas for presenting user interface elements, at least one of which may be used to present the representation of the UDC node. The representation of the UDC node may correspond to the content of the UDC node. For example, if the UDC node represents a nickname for a medication, the representation may include a string that identifies the nickname (e.g., “best toe cream”), an image (e.g., user-captured or obtained from some other source) of the medication, and other information relating to the actual medication (e.g., a proper name for the medication, information about dosage, prescribing doctor). At least some of this information may be obtained from the health record data when the UDC node was generated. Thus, at block 510, the process 500 uses the information stored in the UDC node and logic in the user interface to determine how to present the information from the UDC node.

In some examples, the process 500 may further include the user device 106 establishing a plurality of connections between a plurality of other UDC nodes of the personalized data graph and the UDC node, removing a connection of the plurality of connections between at least one of the other UDC nodes and the UDC node, and responsive to removing the connection, causing the UDC node to sync with the plurality of other UDC nodes.

In some examples, the process 500 may further include the user device 106 generating a concept node of a personalized data graph by at least determining a mapping between a medical concept identified from the health data and a reference node of a reference ontology. In this example, the process 500 may further include the user device 106 receiving a different customization request relating to customizing an aspect of the concept node. In this example, the process 500 may further include the user device 106 generating a different UDC node of the personalized data graph based at least in part on the different customization request. The different UDC node may include different customized information that represents the customized aspect of the concept node. In this example, the process 500 may further include the user device 106 updating, based at least in part on generating the different UDC node, the concept node to include relationship information that represents a relationship between the concept node and the different UDC node.

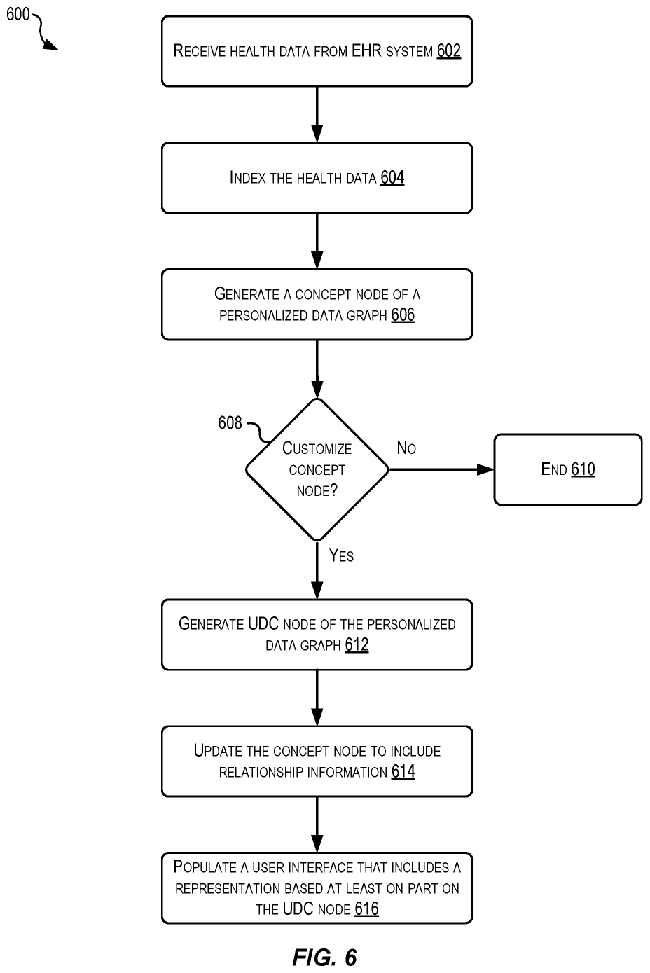

FIG. 6 illustrates a flow chart showing an example process 600 for generating user domain concept nodes of personalized data graphs, according to at least one example. The process 600 may be performed by the health application 222 (FIG. 2) of the user device 106 (FIG. 1). In some examples, at least some portion (or the entirety thereof) may be performed by the service provider 104 (FIG. 1).

The process 600 begins at block 602 by the user device 106 receiving health data from an electronic health record (EHR) system. This block may be performed similar to block 502 of FIG. 5.

At block 604, the process 600 includes the user device 106 indexing the health data. Indexing the health data may include processing the health data to assign a reference coding (e.g., health record identifiers 314) to individual portions of the health data. In some examples, indexing may also include mapping the health data to one or more concepts of the reference ontology concepts (e.g., 304).

At block 606, the process 600 includes the user device 106 generating a concept node of a personalized data graph. This may be performed by at least determining a mapping between a medical concept identified from the health data and a reference node of a reference ontology. In some examples, the concept node generated at block 606 may represent a medical concept in the health data that is, at least at this point, disconnected from a UDC node. Thus, the personalized data graph, which may be stored in the health data database 128 (FIG. 1), may include UDC nodes and non-UDC nodes. A non-UDC node may be a node that represents a medical concept, a medical record, or some other aspect of the health data and that is not customized.

At block 608, the process 600 includes the user device 106 determining whether to customize the concept node created at block 606. In some examples, this may include determining whether a customization request has been received. The customization request may be received via a user interface or other input device, as input by the user. The customization request may relate to customizing an aspect of the concept node. In some examples, this may include determining whether one or more automatic customization triggers have been received. For example, when new health data is processed by the user device 106, this event may trigger the customization of the concept node. As an additional example, when the reference ontology is updated, this may trigger an update to the concept node. In some examples, similar triggers may be associated with updates to UDC nodes. For example, when the underlying health data or reference ontology with which a particular UDC node is associated is updated, the particular UDC node may also be updated.

In some examples, the concept node may represent a specific medication, and the customization request may include a user request to assign a nickname to the specific medication. In this example, the customized information may include the nickname. In some examples, the concept node may represent a specific lab result, and the customization request may include a user request to add the specific lab result to a list. In this example, the customized information may define the list.

If the answer at 608 is “NO,” the process 600 proceeds to 610 and ends. If the answer at 608 is “YES,” at block 612, the process 600 includes the user device 106 generating a user domain concept (UDC) node of the personalized data graph. The generating may be based at least in part on the customization request (or trigger detected at 608). The UDC node may include customized information that represents the customized aspect of the concept node.

At block 614, the process 600 includes the user device 106 updating the concept node to include relationship information. The relationship information may represent a relationship between the concept node and the UDC node. In some examples, the updating may be based at least in part on generating the UDC node. Thus, the concept node may be updated once the UDC node has been generated. In this manner, the concept node may retain information about its relationship with the UDC node. In some examples, the relationship may include at least one of a list member, can support, child, parent, more specific, or less specific.

At block 616, the process 600 includes the user device 106 populating a user interface that includes a representation based at least in part on the UDC node. This may be performed in a manner similar to block 510 of FIG. 5.

In some examples, the process 600 may further include storing the reference ontology in a first storage location on the user device, and storing the personalized data graph in a second storage location on the user device. In some examples, the first storage location (e.g., the ontology database 206) and the second storage location (e.g., the health data database 128) may be logically separate from each other. This may enable updating of one without impacting the other. In some examples, populating the user interface may include, responsive to receiving a request, retrieving, from the second storage location, data associated with the UDC node of the personalized data graph. In this example, populating may also include populating the user interface using the data associated with the UDC node.

In some examples, the customization request may be a first customization request, the aspect may be a first aspect, the concept node may be a first concept node, the UDC node may be a first UDC node, and the customized information may be first customized information. In this example, the process 600 may further include the user device 106 receiving a second customization request relating to customizing a second aspect of a second concept node. The process 600 may further include the user device 106 generating a second UDC node of the personalized data graph based at least in part on the second customization request. The second UDC node may include second customized information that represents the second customized aspect of the second concept node. In some examples, the first and second customization requests may relate to adding the first and second concept nodes to a list. In this example, the representation may include the list generated using information from the first UDC node and the second UDC node.

In some examples, the customization request, which may be analyzed at block 608, may include a request to merge the concept node with a different concept node. In this example, the UDC node may include a single node that represents the concept node and the different concept node. In some examples, the different concept node may represent the medical concept. In some examples, the different concept node may represent a different medical concept.

In some examples, the process 600 may further include the user device 106 updating the reference ontology including the reference node without updating the UDC node of the personalized data graph. In some examples, the process 600 may further include the user device 106 updating the personalized data graph ontology including the UDC node without updating the reference node of the reference ontology.

In some examples, the user device may be a first user device and may be associated with a user profile. In this example, the process 600 may further include the user device 106 receiving, from a service provider, a different personalized data graph generated by a second user device associated with the user profile. The process 600 may additionally include the user device 106 comparing the personalized data graph and the different personalized data graph to generate a combined personalized data graph. The process 600 may additionally include the user device 106 sharing the combined personalized data graph with the service provider.

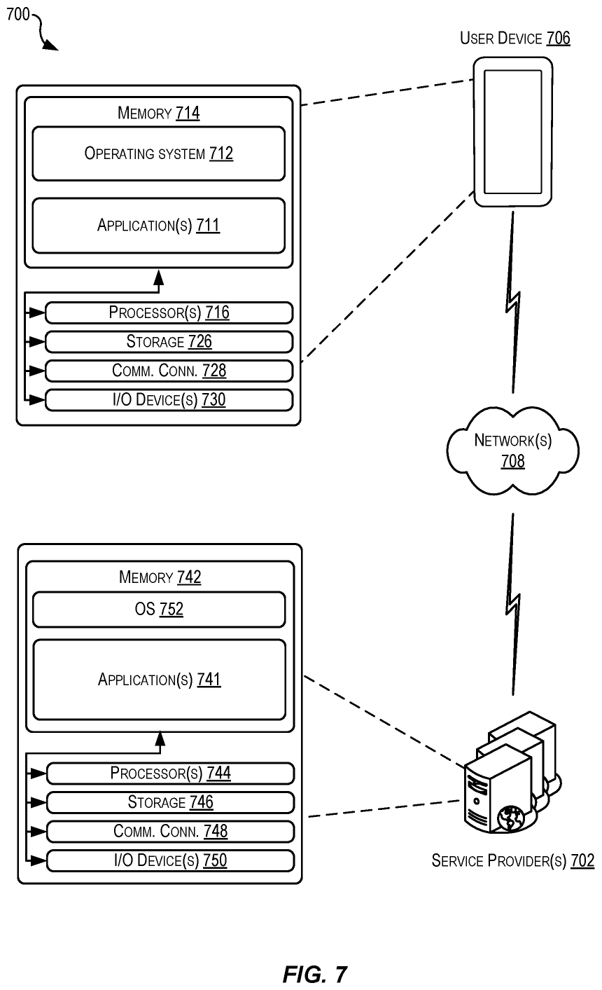

FIG. 7 illustrates an example architecture or environment 700 configured to implement techniques described herein, according to at least one example. In some examples, the example architecture 700 may further be configured to enable a user device 706 and service provider computer 702 to share information. The service provider computer 702 is an example of the service provider 104 and the EHR system 108. The user device 706 is an example of the user device 106. In some examples, the devices may be connected via one or more networks 708 (e.g., via Bluetooth, WiFi, the Internet). In some examples, the service provider computer 702 may be configured to implement at least some of the techniques described herein with reference to the user device 706 and vice versa.

In some examples, the networks 708 may include any one or a combination of many different types of networks, such as cable networks, the Internet, wireless networks, cellular networks, satellite networks, other private and/or public networks, or any combination thereof. While the illustrated example represents the user device 706 accessing the service provider computer 702 via the networks 708, the described techniques may equally apply in instances where the user device 706 interacts with the service provider computer 702 over a landline phone, via a kiosk, or in any other manner. It is also noted that the described techniques may apply in other client/server arrangements (e.g., set-top boxes), as well as in non-client/server arrangements (e.g., locally stored applications, peer-to-peer configurations).

As noted above, the user device 706 may be any type of computing device such as, but not limited to, a mobile phone, a smartphone, a personal digital assistant (PDA), a laptop computer, a desktop computer, a thin-client device, a tablet computer, a wearable device such as a smart watch, or the like. In some examples, the user device 706 may be in communication with the service provider computer 702 via the network 708 or via other network connections.

In one illustrative configuration, the user device 706 may include at least one memory 714 and one or more processing units (or processor(s)) 716. The processor(s) 716 may be implemented as appropriate in hardware, computer-executable instructions, firmware, or combinations thereof. Computer-executable instruction or firmware implementations of the processor(s) 716 may include computer-executable or machine-executable instructions written in any suitable programming language to perform the various functions described. The user device 706 may also include geo-location devices (e.g., a global positioning system (GPS) device or the like) for providing and/or recording geographic location information associated with the user device 706.

The memory 714 may store program instructions that are loadable and executable on the processor(s) 716, as well as data generated during the execution of these programs. Depending on the configuration and type of the user device 706, the memory 714 may be volatile (such as random access memory (RAM)) and/or non-volatile (such as read-only memory (ROM), flash memory). The user device 706 may also include additional removable storage and/or non-removable storage 726 including, but not limited to, magnetic storage, optical disks, and/or tape storage. The disk drives and their associated non-transitory computer-readable media may provide non-volatile storage of computer-readable instructions, data structures, program modules, and other data for the computing devices. In some implementations, the memory 714 may include multiple different types of memory, such as static random access memory (SRAM), dynamic random access memory (DRAM), or ROM. While the volatile memory described herein may be referred to as RAM, any volatile memory that would not maintain data stored therein once unplugged from a host and/or power would be appropriate.

The memory 714 and the additional storage 726, both removable and non-removable, are all examples of non-transitory computer-readable storage media. For example, non-transitory computer-readable storage media may include volatile or non-volatile, removable or non-removable media implemented in any method or technology for storage of information such as computer-readable instructions, data structures, program modules, or other data. The memory 714 and the additional storage 726 are both examples of non-transitory computer-storage media. Additional types of computer-storage media that may be present in the user device 706 may include, but are not limited to, phase-change RAM (PRAM), SRAM, DRAM, RAM, ROM, Electrically Erasable Programmable Read-Only Memory (EEPROM), flash memory or other memory technology, compact disc read-only memory (CD-ROM), digital video disc (DVD) or other optical storage, magnetic cassettes, magnetic tape, magnetic disk storage or other magnetic storage devices, or any other medium that can be used to store the desired information and that can be accessed by the user device 706. Combinations of any of the above should also be included within the scope of non-transitory computer-readable storage media. Alternatively, computer-readable communication media may include computer-readable instructions, program modules, or other data transmitted within a data signal, such as a carrier wave, or other transmission. However, as used herein, computer-readable storage media does not include computer-readable communication media. The memory 714 and/or the additional storage 726 may store the health data database 128 and/or the ontology data database 206.

The user device 706 may also contain communications connection(s) 728 that allow the user device 706 to communicate with a data store, another computing device or server, user terminals, and/or other devices via the network 708. The user device 706 may also include I/O device(s) 730, such as a keyboard, a mouse, a pen, a voice input device, a touch screen input device, a display, speakers, and a printer.

Turning to the contents of the memory 714 in more detail, the memory 714 may include an operating system 712 and/or one or more application programs or services for implementing the features disclosed herein such as applications 711 (e.g., health application 222, digital wallet, third-party applications, browser application, and any other suitable application). In some examples, the applications 711 may include a health application (e.g., the health application 222) to perform similar techniques as described with reference to the user device 106. Similarly, at least some techniques described with reference to the service provider computer 702 may be performed by the user device 106.

The service provider computer 702 may also be any type of computing device such as, but not limited to, a collection of virtual or “cloud” computing resources, a remote server, a mobile phone, a smartphone, a PDA, a laptop computer, a desktop computer, a thin-client device, a tablet computer, a wearable device, a server computer, or a virtual machine instance. In some examples, the service provider computer 702 may be in communication with the user device 706 via the network 708 or via other network connections.

In one illustrative configuration, the service provider computer 702 may include at least one memory 742 and one or more processing units (or processor(s)) 744. The processor(s) 744 may be implemented as appropriate in hardware, computer-executable instructions, firmware, or combinations thereof. Computer-executable instruction or firmware implementations of the processor(s) 744 may include computer-executable or machine-executable instructions written in any suitable programming language to perform the various functions described.

The memory 742 may store program instructions that are loadable and executable on the processor(s) 744, as well as data generated during the execution of these programs. Depending on the configuration and type of service provider computer 702, the memory 742 may be volatile (such as RAM) and/or non-volatile (such as ROM and flash memory). The service provider computer 702 may also include additional removable storage and/or non-removable storage 746 including, but not limited to, magnetic storage, optical disks, and/or tape storage. The disk drives and their associated non-transitory computer-readable media may provide non-volatile storage of computer-readable instructions, data structures, program modules, and other data for the computing devices. In some implementations, the memory 742 may include multiple different types of memory, such as SRAM, DRAM, or ROM. While the volatile memory described herein may be referred to as RAM, any volatile memory that would not maintain data stored therein, once unplugged from a host and/or power, would be appropriate. The memory 742 and the additional storage 746, both removable and non-removable, are both additional examples of non-transitory computer-readable storage media.

The service provider computer 702 may also contain communications connection(s) 748 that allow the service provider computer 702 to communicate with a data store, another computing device or server, user terminals, and/or other devices via the network 708. The service provider computer 702 may also include I/O device(s) 750, such as a keyboard, a mouse, a pen, a voice input device, a touch input device, a display, speakers, and a printer.

Turning to the contents of the memory 742 in more detail, the memory 742 may include an operating system 752 and/or one or more application programs 741 or services for implementing the features disclosed herein including a provider service 210, the provider admin 212, the subscription service 220, business registration system 208, the test harness 216, the device authentication service 226, the data storage engine 228, the data retrieval engine 234, the assets engine 236, the EHR authentication service 238, the logging engine 242, the access control storage 230, health data database 128, and health data storage 232 database, and/or the dashboard 221.

In the following, further clauses are described to facilitate the understanding of the present disclosure.

Clause 1. A computer-implemented method, comprising:
receiving, by a user device, health data from an electronic health record (EHR) system;
receiving a customization request relating to customizing a portion of the health data;
generating a user domain concept (UDC) node of a personalized data graph based at least in part on the customization request, the UDC node comprising customized information that represents the customized portion of the health data;
updating, based at least in part on generating the UDC node, the UDC node to include relationship information that represents a relationship between the UDC node and the portion of the health data; and
populating, at the user device, a user interface that includes a representation of the UDC node.

Clause 2. The computer-implemented method of clause 1, wherein the health data is associated with a user profile of the user device.

Clause 3. The computer-implemented method of any of clauses 1-2, wherein the portion of the health data comprises a plurality of health data samples, and wherein the UDC node comprises individual connections with each of the plurality of health data samples.

Clause 4. The computer-implemented method of any of clauses 1-3, further comprising: after receiving the health data, indexing the health data to assign reference coding to individual portions of the health data, and wherein updating the UDC node to include the relationship information comprises updating the UDC node to include a reference coding assigned to the portion of the health data.

Clause 5. The computer-implemented method of any of clauses 1-4, wherein the customization request comprises at least one of a request to favorite the portion of the health data, a request to give the portion of the health data a nickname, a request to add the portion of the health data to a list, a request to combine the health data with other health data, a request to connect the UDC node with one or more other UDC nodes of the personalized data graph.

Clause 6. The computer-implemented method of any of clauses 1-5, further comprising:
establishing a plurality of connections between a plurality of other UDC nodes of the personalized data graph and the UDC node;
removing a connection of the plurality of connections between at least one of the other UDC nodes and the UDC node; and
responsive to removing the connection, causing the UDC node to sync with the plurality of other UDC nodes.

Clause 7. The computer-implemented method of any of clauses 1-6, further comprising:
generating a concept node of a personalized data graph by at least determining a mapping between a medical concept identified from the health data and a reference node of a reference ontology; and
receiving a different customization request relating to customizing an aspect of the concept node;
generating a different UDC node of the personalized data graph based at least in part on the different customization request, the different UDC node comprising different customized information that represents the customized aspect of the concept node; and
updating, based at least in part on generating the different UDC node, the concept node to include relationship information that represents a relationship between the concept node and the different UDC node.

Clause 8. One or more non-transitory computer-readable media comprising computer-executable instructions that, when executed by one or more processors, cause the one or more processors to perform the method of any one of clauses 1-7.

Clause 9. A computerized system, comprising:
a memory configured to store computer-executable instructions; and
a processor configured to access the memory and execute the computer-executable instructions to perform the method of any one of clauses 1-7.

Clause 10. A computer-implemented method, comprising:
receiving, by a user device, health data from an electronic health record (EHR) system;
generating a concept node of a personalized data graph by at least determining a mapping between a medical concept identified from the health data and a reference node of a reference ontology;
receiving a customization request relating to customizing an aspect of the concept node;
generating a user domain concept (UDC) node of the personalized data graph based at least in part on the customization request, the UDC node comprising customized information that represents the customized aspect of the concept node,
updating, based at least in part on generating the UDC node, the concept node to include relationship information that represents a relationship between the concept node and the UDC node; and
populating, at the user device, a user interface that includes a representation based at least in part on the UDC node.

Clause 11. The computer-implemented method of clause 10, further comprising:
storing the reference ontology in a first storage location on the user device; and
storing the personalized data graph in a second storage location on the user device.

Clause 12. The computer-implemented method of clause 11, wherein populating the user interface comprises, responsive to receiving a request, retrieving, from the second storage location, data associated with the UDC node of the personalized data graph; and populating the user interface using the data associated with the UDC node.

Clause 13. The computer-implemented method of any of clauses 10-12, wherein the concept node represents a specific medication and the customization request comprises a user request to assign a nickname to the specific medication, and wherein the customized information comprises the nickname.

Clause 14. The computer-implemented method of any of clauses 10-13, wherein the concept node represents a specific lab result and the customization request comprises a user request to add the specific lab result to a list, and wherein the customized information defines the list.

Clause 15. The computer-implemented method of any of clauses 10-14, wherein the customization request is a first customization request, the aspect is a first aspect, the concept node is a first concept node, the UDC node is a first UDC node, and the customized information is first customized information, the method further comprising:
receiving a second customization request relating to customizing a second aspect of a second concept node; and
generating a second UDC node of the personalized data graph based at least in part on the second customization request, the second UDC node comprising second customized information that represents the customized second aspect of the second concept node.

Clause 16. The computer-implemented method of clause 15, wherein the first and second customization requests relate to adding the first and second concept nodes to a list, and wherein the representation comprises the list generated using information from the first UDC node and the second UDC node.

Clause 17. The computer-implemented method of any of clauses 10-16, wherein the customization request comprises a request to merge the concept node with a different concept node, wherein the UDC node comprises a single node that represents the concept node and the different concept node.

Clause 18. The computer-implemented method of clause 17, wherein the different concept node represents the medical concept.

Clause 19. The computer-implemented method of clause 17, wherein the different concept nodes represents a different medical concept.

Clause 20. The computer-implemented method of any of clauses 10-19, wherein the relationship comprises at least one of a list member, can support, child, parent, more specific, or less specific.

Clause 21. The computer-implemented method of any of clauses 10-20, further comprising updating the reference ontology including the reference node without updating the UDC node of the personalized data graph.

Clause 22. The computer-implemented method of any of clauses 10-21, further comprising updating the personalized data graph including the UDC node without updating the reference node of the reference ontology.

Clause 23. The computer-implemented method of any of clauses 10-22, wherein the user device is a first user device and is associated with a user profile, further comprising receiving, from a service provider, a different personalized data graph generated by a second user device associated with the user profile;
comparing the personalized data graph and the different personalized data graph to generate a combined personalized data graph; and
sharing the combined personalized data graph with the service provider.

Clause 24. One or more non-transitory computer-readable media comprising computer-executable instructions that, when executed by one or more processors, cause the one or more processors to perform the method of any one of clauses 10-23.

Clause 25. A computerized system, comprising:
a memory configured to store computer-executable instructions; and
a processor configured to access the memory and execute the computer-executable instructions to perform the method of any one of clauses 10-23

The various examples can be further implemented in a wide variety of operating environments, which in some cases can include one or more user computers, computing devices, or processing devices which can be used to operate any of a number of applications. User or client devices can include any of a number of general purpose personal computers, such as desktop or laptop computers running a standard operating system, as well as cellular, wireless, and handheld devices running mobile software and capable of supporting a number of networking and messaging protocols. Such a system also can include a number of workstations running any of a variety of commercially-available operating systems and other known applications for purposes such as development and database management. These devices also can include other electronic devices, such as dummy terminals, thin-clients, gaming systems, and other devices capable of communicating via a network.

Most examples utilize at least one network that would be familiar to those skilled in the art for supporting communications using any of a variety of commercially-available protocols, such as TCP/IP, OSI, FTP, UPnP, NFS, CIFS, and AppleTalk. The network can be, for example, a local area network, a wide-area network, a virtual private network, the Internet, an intranet, an extranet, a public switched telephone network, an infrared network, a wireless network, and any combination thereof.

In examples utilizing a network server, the network server can run any of a variety of server or mid-tier applications, including HTTP servers, FTP servers, CGI servers, data servers, Java servers, and business application servers. The server(s) may also be capable of executing programs or scripts in response to requests from user devices, such as by executing one or more applications that may be implemented as one or more scripts or programs written in any programming language, such as Java®, C, C# or C++, or any scripting language, such as Perl, Python, or TCL, as well as combinations thereof. The server(s) may also include database servers, including without limitation those commercially available from Oracle®, Microsoft®, Sybase®, and IBM®.

The environment can include a variety of data stores and other memory and storage media as discussed above. These can reside in a variety of locations, such as on a storage medium local to (and/or resident in) one or more of the computers or remote from any or all of the computers across the network. In a particular set of examples, the information may reside in a storage-area network (SAN) familiar to those skilled in the art. Similarly, any necessary files for performing the functions attributed to the computers, servers, or other network devices may be stored locally and/or remotely, as appropriate. Where a system includes computerized devices, each such device can include hardware elements that may be electrically coupled via a bus, the elements including, for example, at least one central processing unit (CPU), at least one input device (e.g., a mouse, keyboard, controller, touch screen, keypad), and at least one output device (e.g., a display device, printer, speaker). Such a system may also include one or more storage devices, such as disk drives, optical storage devices, and solid-state storage devices such as RAM or ROM, as well as removable media devices, memory cards, or flash cards.

Such devices can also include a computer-readable storage media reader, a communications device (e.g., a modem, a network card (wireless or wired), an infrared communication device), and working memory as described above. The computer-readable storage media reader can be connected with, or configured to receive, a non-transitory computer-readable storage medium, representing remote, local, fixed, and/or removable storage devices as well as storage media for temporarily and/or more permanently containing, storing, transmitting, and retrieving computer-readable information. The system and various devices also typically will include a number of software applications, modules, services, or other elements located within at least one working memory device, including an operating system and application programs, such as a client application or browser. It should be appreciated that alternate examples may have numerous variations from that described above. For example, customized hardware might also be used and/or particular elements might be implemented in hardware, software (including portable software, such as applets), or both. Further, connection to other computing devices such as network input/output devices may be employed.

Non-transitory storage media and computer-readable media for containing code, or portions of code, can include any appropriate media known or used in the art, including storage media, such as, but not limited to, volatile and non-volatile, removable and non-removable media implemented in any method or technology for storage of information such as computer-readable instructions, data structures, program modules, or other data, including RAM, ROM, EEPROM, flash memory or other memory technology, CD-ROM, DVD or other optical storage, magnetic cassettes, magnetic tape, magnetic disk storage or other magnetic storage devices, or any other medium which can be used to store the desired information and which can be accessed by a system device. Based at least in part on the disclosure and teachings provided herein, a person of ordinary skill in the art will appreciate other ways and/or methods to implement the various examples.

The specification and drawings are, accordingly, to be regarded in an illustrative rather than a restrictive sense. It will, however, be evident that various modifications and changes may be made thereunto without departing from the broader spirit and scope of the disclosure as set forth in the claims.

Other variations are within the spirit of the present disclosure. Thus, while the disclosed techniques are susceptible to various modifications and alternative constructions, certain illustrated examples thereof are shown in the drawings and have been described above in detail. It should be understood, however, that there is no intention to limit the disclosure to the specific form or forms disclosed, but on the contrary, the intention is to cover all modifications, alternative constructions and equivalents falling within the spirit and scope of the disclosure, as defined in the appended claims.

The use of the terms “a” and “an” and “the” and similar referents in the context of describing the disclosed examples (especially in the context of the following claims) are to be construed to cover both the singular and the plural, unless otherwise indicated herein or clearly contradicted by context. The terms “comprising,” “having,” “including,” and “containing” are to be construed as open-ended terms (e.g., meaning “including, but not limited to”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate examples of the disclosure and does not pose a limitation on the scope of the disclosure unless otherwise claimed. No language in the specification should be construed as indicating any non-claimed element as essential to the practice of the disclosure.

Disjunctive language such as the phrase “at least one of X, Y, or Z,” unless specifically stated otherwise, is otherwise understood within the context as used in general to present that an item, term, etc., may be either X, Y, or Z, or any combination thereof (e.g., X, Y, and/or Z). Thus, such disjunctive language is not generally intended to, and should not, imply that certain examples require at least one of X, at least one of Y, or at least one of Z to each be present.

Preferred examples of this disclosure are described herein, including the best mode known to the inventors for carrying out the disclosure. Variations of those preferred examples may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

As described above, one aspect of the present technology is the gathering and use of data available from various sources to provide a comprehensive and complete window to a user's personal health record. The present disclosure contemplates that in some instances, this gathered data may include personally identifiable information (PII) data that uniquely identifies or can be used to contact or locate a specific person. Such personal information data can include demographic data, location-based data, telephone numbers, email addresses, Twitter IDs, home addresses, data or records relating to a user's health or level of fitness (e.g., vital sign measurements, medication information, exercise information), date of birth, health record data, or any other identifying or personal or health information.

The present disclosure recognizes that the use of such personal information data, in the present technology, can be used to the benefit of users. For example, the personal information data can be used to provide enhancements to a user's personal health record. Further, other uses for personal information data that benefit the user are also contemplated by the present disclosure. For instance, health and fitness data may be used to provide insights into a user's general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the U.S., collection of or access to certain health data may be governed by federal and/or state laws, such as the Health Insurance Portability and Accountability Act (HIPAA); whereas health data in other countries may be subject to other regulations and policies and should be handled accordingly. Hence, different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of advertisement delivery services or other services relating to health record management, the present technology can be configured to allow users to select to “opt in” or “opt out” of participation in the collection of personal information data during registration for services or anytime thereafter. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health-related applications, data de-identification can be used to protect a user's privacy. De-identification may be facilitated, when appropriate, by removing specific identifiers (e.g., date of birth), controlling the amount or specificity of data stored (e.g., collecting location data at a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data.

What is claimed is:

1. A computer-implemented method, comprising:
receiving, by a user device, health data from an electronic health record (EHR) system;
receiving a customization request relating to customizing a portion of the health data;
generating a user domain concept (UDC) node of a personalized data graph based at least in part on the customization request, the UDC node comprising customized information that represents the customized portion of the health data;
updating, based at least in part on generating the UDC node, the UDC node to include relationship information that represents a relationship between the UDC node and the portion of the health data; and
populating, at the user device, a user interface that includes a representation of the UDC node.

2. The computer-implemented method of claim 1, wherein the health data is associated with a user profile of the user device.

3. The computer-implemented method of claim 1, wherein the portion of the health data comprises a plurality of health data samples, and wherein the UDC node comprises individual connections with each of the plurality of health data samples.

4. The computer-implemented method of claim 1, further comprising:
after receiving the health data, indexing the health data to assign reference coding to individual portions of the health data, and wherein updating the UDC node to include the relationship information comprises updating the UDC node to include a reference coding assigned to the portion of the health data.

5. The computer-implemented method of claim 1, wherein the customization request comprises at least one of a request to favorite the portion of the health data, a request to give the portion of the health data a nickname, a request to add the portion of the health data to a list, a request to combine the health data with other health data, a request to connect the UDC node with one or more other UDC nodes of the personalized data graph.

6. The computer-implemented method of claim 1, further comprising:
establishing a plurality of connections between a plurality of other UDC nodes of the personalized data graph and the UDC node;
removing a connection of the plurality of connections between at least one of the other UDC nodes and the UDC node; and
responsive to removing the connection, causing the UDC node to sync with the plurality of other UDC nodes.

7. The computer-implemented method of claim 1, further comprising:
generating a concept node of a personalized data graph by at least determining a mapping between a medical concept identified from the health data and a reference node of a reference ontology; and
receiving a different customization request relating to customizing an aspect of the concept node;
generating a different UDC node of the personalized data graph based at least in part on the different customization request, the different UDC node comprising different customized information that represents the customized aspect of the concept node; and
updating, based at least in part on generating the different UDC node, the concept node to include relationship information that represents a relationship between the concept node and the different UDC node.

8. One or more non-transitory computer-readable media comprising computer-executable instructions that, when executed by one or more processors of a user device, cause the user device to perform operations comprising:
receiving, by the user device, health data from an electronic health record (EHR) system;
receiving a customization request relating to customizing a portion of the health data;
generating a user domain concept (UDC) node of a personalized data graph based at least in part on the customization request, the UDC node comprising customized information that represents the customized portion of the health data;
updating, based at least in part on generating the UDC node, the UDC node to include relationship information that represents a relationship between the UDC node and the portion of the health data; and
populating, at the user device, a user interface that includes a representation of the UDC node.

9. The one or more non-transitory computer-readable media of claim 8, wherein the health data is associated with a user profile of the user device.

10. The one or more non-transitory computer-readable media of claim 8, wherein the portion of the health data comprises a plurality of health data samples, and wherein the UDC node comprises individual connections with each of the plurality of health data samples.

11. The one or more non-transitory computer-readable media of claim 8, further comprising additional computer-executable instructions that, when executed by the one or more processors, cause the user device to perform additional operations comprising after receiving the health data, indexing the health data to assign reference coding to individual portions of the health data, and wherein updating the UDC node to include the relationship information comprises updating the UDC node to include a reference coding assigned to the portion of the health data.

12. The one or more non-transitory computer-readable media of claim 8, wherein the customization request comprises at least one of a request to favorite the portion of the health data, a request to give the portion of the health data a nickname, a request to add the portion of the health data to a list, a request to combine the health data with other health data, a request to connect the UDC node with one or more other UDC nodes of the personalized data graph.

13. The one or more non-transitory computer-readable media of claim 8, further comprising additional computer-executable instructions that, when executed by the one or more processors, cause the user device to perform additional operations comprising:
establishing a plurality of connections between a plurality of other UDC nodes of the personalized data graph and the UDC node;
removing a connection of the plurality of connections between at least one of the other UDC nodes and the UDC node; and
responsive to removing the connection, causing the UDC node to sync with the plurality of other UDC nodes.

14. The one or more non-transitory computer-readable media of claim 8, further comprising additional computer-executable instructions that, when executed by the one or more processors, cause the user device to perform additional operations comprising:
generating a concept node of a personalized data graph by at least determining a mapping between a medical concept identified from the health data and a reference node of a reference ontology; and
receiving a different customization request relating to customizing an aspect of the concept node;
generating a different UDC node of the personalized data graph based at least in part on the different customization request, the different UDC node comprising different customized information that represents the customized aspect of the concept node; and
updating, based at least in part on generating the different UDC node, the concept node to include relationship information that represents a relationship between the concept node and the different UDC node.

15. A user device, comprising:
a memory configured to store computer-executable instructions; and
a processor configured to access the memory and execute the computer-executable instructions to at least:
receive health data from an electronic health record (EHR) system;
receive a customization request relating to customizing a portion of the health data;
generate a user domain concept (UDC) node of a personalized data graph based at least in part on the customization request, the UDC node comprising customized information that represents the customized portion of the health data;
update, based at least in part on generating the UDC node, the UDC node to include relationship information that represents a relationship between the UDC node and the portion of the health data; and
populate a user interface that includes a representation of the UDC node.

16. The user device of claim 15, wherein the health data is associated with a user profile of the user device.

17. The user device of claim 15, wherein the portion of the health data comprises a plurality of health data samples, and wherein the UDC node comprises individual connections with each of the plurality of health data samples.

18. The user device of claim 15, wherein the memory comprises additional computer-executable instructions, and the processor is further configured to execute the additional computer-executable instructions to at least, after receiving the health data, index the health data to assign reference coding to individual portions of the health data, and wherein updating the UDC node to include the relationship information comprises updating the UDC node to include a reference coding assigned to the portion of the health data.

19. The user device of claim 15, wherein the memory comprises additional computer-executable instructions, and the processor is further configured to execute the additional computer-executable instructions to at least:
establish a plurality of connections between a plurality of other UDC nodes of the personalized data graph and the UDC node;
remove a connection of the plurality of connections between at least one of the other UDC nodes and the UDC node; and
responsive to removing the connection, cause the UDC node to sync with the plurality of other UDC nodes.

20. The user device of claim 15, wherein the memory comprises additional computer-executable instructions, and the processor is further configured to execute the additional computer-executable instructions to at least:
generating a concept node of a personalized data graph by at least determining a mapping between a medical concept identified from the health data and a reference node of a reference ontology; and
receiving a different customization request relating to customizing an aspect of the concept node;
generating a different UDC node of the personalized data graph based at least in part on the different customization request, the different UDC node comprising different customized information that represents the customized aspect of the concept node; and
updating, based at least in part on generating the different UDC node, the concept node to include relationship information that represents a relationship between the concept node and the different UDC node.
